(19) United States
(12) Patent Application Publication
(10) Pub. No.: US 2025/0194661 A1
(43) Pub. Date: Jun. 19, 2025

(54) COMPOSITIONS FOR DELIVERY OF NICOTINE

(71) Applicant: PNEUMA RESPIRATORY, INC., Boone, NC (US)

(72) Inventor: Matthew Culpepper, Boone, NC (US)

(73) Assignee: PNEUMA RESPIRATORY, INC., Boone, NC (US)

(21) Appl. No.: 18/986,462

(22) Filed: Dec. 18, 2024

Related U.S. Application Data
(63) Continuation of application No. PCT/US2024/031401, filed on May 29, 2024.
(60) Provisional application No. 63/470,254, filed on Jun. 1, 2023.

(51) Int. Cl. A24B 15/167; A24B 15/30; A24B 15/32; A24B 15/40; A24F 40/05; A24F 40/10; A24F 40/485; A61K 9/00; A61K 31/465

(52) U.S. Cl. CPC A24B 15/167; A24B 15/302; A24B 15/32; A24B 15/403; A24F 40/05; A24F 40/10; A24F 40/485; A61K 9/0078; A61K 31/465

(57) ABSTRACT

Embodiments of the present disclosure provide compositions and methods for administering nicotine to the respiratory system of a subject. Accordingly, embodiments herein include compositions comprising nicotine that includes nicotine in combination with L-lactic acid and water. Embodiments herein also include methods of making compositions herein, methods of delivering compositions herein, and methods of treating a subject by administering one or more compositions herein.

### CROSS-REFERENCE TO RELATED APPLICATION

This application is a continuation of International Application No. PCT/US2024/031401 filed May 29, 2024, which claims benefit of priority to U.S. Provisional Patent Application Ser. No. 63/470,254, filed Jun. 1, 2023, and titled, “COMPOSITIONS FOR DELIVERY OF NICOTINE,” the contents of all of which are incorporated herein by reference in their entirety.

### FIELD

Embodiments of the instant disclosure generally relate to compositions and methods for administering nicotine and nicotine-containing compositions to the respiratory system.

### BACKGROUND

In nature chiral products are usually produced in optically pure forms by an organism. Frequently one isomer is harmless while the other optical form could be toxic and/or interfere crucial pathways in the body. However, some isomers do not exert significant problems. Although optical isomers (enantiomers) have been known for a long time, laboratory testing is elaborate and expensive. (See, e.g., Biomed Res Int. Jun. 17, 2020 “D-Lactic as a Metabolite: Toxicology, Diagnosis, and Detection”, incorporated by reference in its entirety.)

L-lactate is found in human blood at a range of 0.5 to 1 mmol/L. D-lactic acid is not naturally found in the blood or involved in basic metabolic processes of most life forms. In conclusion, D-lactic acid is not a highly toxic compound however it is a toxic metabolite that can cause health problems and complicate other pathologies. However, further research is still required.

### SUMMARY

Embodiments of the present disclosure relate to methods and compositions for administering at nicotine to the respiratory system of a subject. In certain embodiments, the present disclosure provides compositions that may comprise nicotine, lactic acid, water, or any combination thereof. In certain embodiments, the compositions may further comprise ethanol and/or menthol.

In some aspects, the lactic acid comprises a racemic mixture of L-lactic acid and D-lactic acid.

In some aspects, the lactic acid comprises L-lactic acid. In some aspects, the composition consists essentially of L-lactic acid and contains no detectable amounts of D-lactic acid. In some aspects, the composition consists essentially of L-lactic acid and contains only trace amounts of D-lactic acid.

In some embodiments, the compositions herein do not contain D-lactic acid. In some embodiments, the compositions herein contain only trace amounts of D-lactic acid. In some embodiments, the compositions herein contain undetectable amounts of D-lactic acid.

In some embodiments, compositions herein may comprise nicotine in a concentration of about 0.1% (w/w) to about 5% (w/w). In some aspects, the compositions herein may comprise about 1.8% (w/w) to about 2.2% (w/w) nicotine. In some aspects, the compositions herein may comprise about 2.0% (w/w) nicotine.

In some embodiments, compositions herein may comprise lactic acid in a concentration of about 0.1% (w/w) to about 5% (w/w). In some aspects, the compositions herein may comprise about 1.6% (w/w) to about 2.0% (w/w) lactic acid. In some aspects, the compositions herein may comprise about 1.8% (w/w) lactic acid.

In some embodiments, compositions herein may comprise L-lactic acid in a concentration of about 0.1% (w/w) to about 5% (w/w). In some aspects, the compositions herein may comprise about 1.6% (w/w) to about 2.0% (w/w) L-lactic acid. In some aspects, the compositions herein may comprise about 1.8% (w/w) L-lactic acid. In some aspects, the compositions do not contain D-lactic acid or do not contain detectable amounts of D-lactic acid.

In some embodiments, compositions herein may comprise water. In some embodiments, compositions herein may comprise water wherein the water may be type I water, deionized water, distilled water, or a combination thereof. In some aspects, the water may be type I water.

In some embodiments, composition herein may comprise ethanol. The ethanol may be at a concentration of about ≤10% (w/w).

In some embodiments, composition herein may comprise propylene glycol. The propylene may be at a concentration of about ≤15% (w/w).

In some embodiments, composition herein may comprise menthol. The menthol may be at a concentration of about ≤0.9% (w/w). The menthol may be L-menthol.

In some embodiments, compositions herein may be a composition for inhalation by a subject. In some embodiments, compositions herein may be delivered to the respiratory system of a subject.

In certain embodiments, the present disclosure provides methods of making any one of the compositions disclosed herein.

In certain embodiments, the present disclosure provides methods of delivering any one of the compositions disclosed herein to the respiratory system of a user. In some embodiments, methods herein may deliver a composition disclosed herein as an ejected stream of droplets and vapor in a respirable range to the respiratory system of a user. In other embodiments, methods herein may deliver a composition disclosed herein as an ejected stream of droplets without vapor in a respirable range to the respiratory system of a user.

In some embodiments, methods herein may deliver a composition comprising nicotine to a user. In some embodiments, methods herein may deliver a composition comprising nicotine to a user to treat or ameliorate one or more diseases, conditions or disorders.

In some embodiments, a method of delivering a composition disclosed herein as an ejected stream of droplets and vapor is provided. In other embodiments, methods herein may deliver a composition disclosed herein as an ejected stream of droplets without vapor in a respirable range to the respiratory system of a user.

In some embodiments, the composition may include cyclodextrin to aid in solubilization of flavoring components. The concentration of cyclodextrin may be ≤5% (w/w).

In some embodiments, composition herein may comprise of a cooling agent WS-23 (2-isopropyl-N,2,3-trimethylbutyramide). The WS-23 may be at a concentration of about ≤0.5% (w/w).In some embodiments, composition herein may comprise of a concentrated fruit juices. The process of fruit extraction is removing all the water from the juice by heat, and filtering the material left over. Another method would be do use an organic solvent such as, but not limited to ethanol, supercritical carbon dioxide, glycerin, etc. These extractions can be increased by using sonication or refluxing the fruit in the solvents. Once the solvent has extracted the fruit flavor compounds, it can be removed by evaporation and/or liquid-liquid extraction using water as the final solvent. The concentration of the concentrated fruit extract of about ≤5% (w/w).

Terms, unless defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” are used interchangeably herein and refer to any mammalian subject for whom diagnosis, treatment, prophylaxis or therapy is desired, particularly humans.

As used herein, “treat,” “treating” or “treatment” can refer to treating, reversing, ameliorating, or inhibiting onset or inhibiting progression of a health condition or disease or a symptom of the health condition or disease.

Unless otherwise defined, all technical terms used herein have the same meaning as commonly understood by one of ordinary skill in the art to which this disclosure belongs.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

In certain aspects, the present disclosure generally relates to methods for delivering a fluid composition comprising nicotine as an ejected stream of droplets in a respirable range to the respiratory system of a user. In certain aspects, the fluid composition comprising nicotine may be delivered at a high dose concentration and efficacy, as compared to alternative dosing routes and standard inhalation technologies. In some aspects, the fluid composition comprising nicotine may be delivered to the user with lower levels of contaminants, undesired compounds, etc. as compared to alternative dosing routes and standard inhalation technologies. In some aspects, the fluid composition comprising nicotine may comprise L-lactic acid. In some aspects, the fluid composition comprising nicotine may comprise no detectable or only trace amounts of D-lactic acid.

### I. Compositions

Certain embodiments herein provided for compositions comprising nicotine. In some embodiments, compositions herein may comprise at nicotine, lactic acid, water, or any combination thereof. In some embodiments, the lactic acid is L-lactic acid. In some embodiments the compositions further comprise ethanol and/or menthol. In some embodiments, compositions herein may be solutions for delivery of nicotine to the respiratory system of a subject.

In some embodiments, compositions disclosed herein may encompass nicotine.

In some embodiments, compositions disclosed herein may encompass about 1% to about 80%, about 5% to about 50%, or about 10% to about 40% nicotine by weight of the composition. In some embodiments, compositions disclosed herein may encompass about 0.1%, about 1%, about 5%, about 10%, about 20%, about 30%, about 40%, about 50%, about 60%, about 70%, or about 80% (w/w) nicotine of the composition. In some embodiments, compositions disclosed herein may encompass about 0.1% (w/w) to about 5% (w/w) nicotine. In some embodiments, compositions disclosed herein may encompass about 0.1% (w/w) to about 5% (w/w) nicotine. In some aspects, the compositions herein may comprise about 1.8% (w/w) to about 2.2% (w/w) nicotine. In some aspects, the compositions herein may comprise about 2.0% (w/w) nicotine.

In some embodiments, compositions disclosed herein may comprise nicotine having at least about 85% purity. In some embodiments, compositions disclosed herein may comprise nicotine having about 85% to 99% (e.g., about 85%, 90%, 95%, 96%, 97%, 98%, 99%) purity. In some embodiments, compositions disclosed herein may comprise at least pure nicotine. Purity of nicotine herein may be determined using methods known in the art, including but not limited to high-performance liquid chromatography (HPLC).

In some embodiments, compositions disclosed herein may encompass nicotine in a concentration of about 0.1 mg/mL to about 20 mg/mL (e.g., about 0.1, 0.5, 1, 2, 3, 4, 5, 6, 7, 8, 9, 10, 11, 12, 13, 14, 15, 16, 17, 18, 19, 20 mg/mL).

In some embodiments, composition disclosed herein comprise lactic acid. In some embodiments, the lactic acid is L-lactic acid (L-(+)-lactic acid).

In some embodiments, compositions disclosed herein may encompass about 1% to about 80%, about 5% to about 50%, or about 10% to about 40% (w/w) L-lactic acid of the composition. In some embodiments, compositions disclosed herein may encompass about 0.1%, about 1%, about 5%, about 10%, about 20%, about 30%, about 40%, about 50%, about 60%, about 70%, or about 80% (w/w) L-lactic acid of the composition. In some embodiments, compositions disclosed herein may encompass about 0.1% (w/w) to about 5% (w/w) L-lactic acid. In some embodiments, compositions disclosed herein may encompass about 0.1% (w/w) to about 5% (w/w) L-lactic acid. In some aspects, the compositions herein may comprise about 1.6% (w/w) to about 2.0% (w/w) L-lactic acid. In some aspects, the compositions herein may comprise about 1.8% (w/w) L-lactic acid.

In some embodiments, the compositions herein do not contain D-lactic acid. In some embodiments, the compositions herein contain only trace amounts of D-lactic acid. In some embodiments, the compositions herein contain no detectable amounts of D-lactic acid. In some embodiments, the compositions herein contain lactic acid wherein the lactic acid essentially consists of L-lactic acid.

In some embodiments, compositions herein may include water. In some embodiments, compositions herein may include deionized water. In some embodiments, compositions herein may include distilled water. In some embodiments, compositions herein may include Type I water. Type I water is defined by the American Society for Testing and Materials (ASTM) as having a resistivity of >18 MΩ-cm, a conductivity of <0.056 μS/cm and <50 ppb of Total Organic Carbons (TOC).

In some embodiments, the compositions disclosed herein further comprise ethanol. The terms “ethanol” and “ethyl alcohol” are understood to have the same meaning and are used interchangeably herein. In some embodiments, compositions herein may include ethanol having a proof of about 180 to about 200 (e.g., about 180, 185, 190, 195, 200). Proof as used herein is defined as twice the alcohol (ethanol) content by volume. As an example, an ethanol having 180 proof comprises about 90% ethanol whereas an ethanol having 200 proof comprises about 100% (i.e., ≥99.5%) ethanol. In some embodiments, compositions herein may include pure ethanol (i.e., 200 proof ethanol). In some embodiments, compositions herein may include ethanol having a proof of about 200.

In some embodiments, compositions disclosed herein may encompass about 1% to about 95%, about 5% to about 50%, or about 10% to about 40% ethanol (w/w) of the composition. In some embodiments, compositions disclosed herein may encompass about 0.1%, about 1%, about 5%, about 10%, about 20%, about 30%, about 40%, about 50%, about 60%, about 70%, about 80%, about 90%, or about 95% (w/w) ethanol of the composition. In some embodiments, compositions disclosed herein may encompass about 0.1% (w/w) to about 20% (w/w) ethanol. In some embodiments, compositions disclosed herein may encompass about 1% (w/w) to about 15% (w/w) ethanol. In some aspects, the compositions herein may comprise about 10% (w/w) ethanol.

In some embodiments, compositions disclosed herein may also comprise menthol. The menthol may be L-menthol.

In some embodiments, compositions disclosed herein may encompass about 0.01% (w/w) to about 95% (w/w) menthol of the composition. In some embodiments, compositions disclosed herein may encompass about 0.01%, about 0.02%, about 0.03%, about 0.04%, about 0.05%, about 0.06%, about 0.07%, about 0.08%, about 0.09%, about 0.1%, about 1%, about 5%, about 10%, about 20%, about 30%, about 40%, about 50%, about 60%, about 70%, about 80%, about 90%, or about 95% (w/w) menthol of the composition. In some embodiments, compositions disclosed herein may encompass about 0.01% (w/w) to about 0.1% (w/w) menthol. In some embodiments, compositions disclosed herein may encompass about 0.05% (w/w) to about 0.1% (w/w) menthol. In some aspects, the compositions herein may comprise about 0.09% (w/w) menthol.

In some embodiments, compositions herein may encompass at least one or more additional pharmaceutically acceptable carrier or diluent. In some embodiments, compositions or solutions herein may further include various emulsifiers, surfactants, solubilizers, stabilizers, flavors, and other pharmaceutically acceptable carriers suitable for delivery to the respiratory system. In accordance with these embodiments, pharmaceutically acceptable carriers and/or diluents for use in compositions herein diluent can be a liquid carrier or diluent comprising at least one of water, propylene glycol and pharmaceutically acceptable fluids. Pharmaceutically acceptable fluids for use herein can include, but are not limited to, polar solvents, including, but not limited to, compounds that contain hydroxyl groups or other polar groups. Solvents for use herein can include, but are not limited to, water or alcohols, such as ethanol, isopropanol, and glycols including propylene glycol, polyethylene glycol, polypropylene glycol, glycol ether, glycerol and polyoxyethylene alcohols. Polar solvents can also include protic solvents, including, but not limited to, water, aqueous saline solutions with one or more pharmaceutically acceptable salt(s), alcohols, glycols or a mixture there of. In some embodiments, water for use in the present compositions can meet or exceed the applicable regulatory requirements for use in inhaled drugs.

In some embodiments, composition herein may have a pH of about 4-6. In some embodiments, the pH is about 4.5 to about 5.5. In some embodiments, the pH is about 5.0 to about 5.4. In some embodiments, the pH is about 5.2.

In some embodiments, compositions herein may be a solution. In some embodiments, compositions herein may be an organic solution or an aqueous solution. In some embodiments, compositions herein may be a solution for inhalation by a subject. In some embodiments, compositions herein may be a solution for delivery of nicotine to the respiratory system of a subject. In some embodiments, compositions herein may be a solution for intranasal administration. In some embodiments, compositions herein may not be a suspension.

In some embodiments, compositions herein may include one or more nicotine, L-lactic acid, and/or water. The composition may comprise 2% (w/w) nicotine, about 1.8% (w/w) L-lactic acid, and type I water. In some embodiments, the composition may further comprise ethanol and/or menthol. In some embodiments, the composition may comprise about 10% (w/w) of ethanol and/or about 0.9% (w/w) of menthol. In some embodiments, the composition does not comprise D-lactic acid. In some embodiments, the pH of the composition is about 5.2.

### II. Methods

Effective and efficient delivery of substances to the respiratory system of a user, and the synchronization of the administration of substances to the respiratory system of the user with the inspiration/expiration cycle of the user has always posed a problem. For instance, optimum deposition in alveolar airways generally requires droplets with aerodynamic diameters in the ranges of 1 to 6 μm, with droplets below about 4 μm shown to more effectively reach the alveolar region of the lungs and larger droplets above about 6 μm shown to typically deposited on the tongue or strike the throat and coat the bronchial passages. Smaller droplets, for example less than about 1 μm, penetrate more deeply into the lungs and have a tendency to be exhaled. As such, methods for delivering a fluid composition comprising nicotine as an ejected stream of droplets in a respirable range in accordance with aspects of the disclosure requires the ability to precisely target droplet sizes for the particular use.

In certain embodiments, the present disclosure provides methods for delivering a fluid composition comprising nicotine as an ejected stream of droplets in a respirable range in accordance with aspects of the disclosure requires the ability to precisely target droplet sizes for the particular use. In accordance with certain aspects of the disclosure, effective deposition of an ejected stream of droplets of a fluid composition comprising nicotine into the lungs of a user generally requires droplets less than about 5-6 μm, e.g., less than about 3.2 μm, in diameter. Without intending to be limited by theory, to deliver an ejected stream of droplets to the lungs, a droplet delivery device must impart a momentum that is sufficiently high to permit ejection out of the device, but sufficiently low to prevent deposition on the tongue or in the back of the throat. Droplets below approximately 5-6 μm in diameter are transported almost completely by motion of the airstream and entrained air that carry them and not by their own momentum.

In certain embodiments, the methods of the present disclosure result in minimal or no mouth or throat irritation. In certain embodiments, the methods include generating an ejected stream of droplets of a fluid composition comprising nicotine with coordinated and precise timing during a user's inspiration cycle to as to maximize delivery into the respiratory system, while minimizing or eliminating mouth or throat irritation. Without intending to be limited by theory, as described herein, the small droplets generated via the methods of the disclosure are transported almost completely by motion of airstream and entrained air. Using this entrained motion and tuned droplet size, the ejection of droplets may be focused so as to eject during peak flow of the inspiration cycle so as to optimize inhalation into the target site in the respiratory system (e.g., deep lungs), while minimizing inadvertent delivery to non-desired sites in the respiratory system (e.g., mouth and throat).

As discussed above, effective delivery of droplets deep into the lung airways require droplets that are less than about 5-6 microns in diameter, specifically droplets with mass mean aerodynamic diameters (MMAD) that are less than about 5 microns. However, for certain agents and uses, droplets about 1 μm or smaller for quick adsorption in the deep lung may be desirable, e.g., it may be desired to utilize droplets less than 4 μm, less than 3.2 μm, less than 3 μm, less than 2 μm, and less than 1 μm for the delivery nicotine to the deep lungs. The mass mean aerodynamic diameter is defined as the diameter at which 50% of the droplets by mass are larger and 50% are smaller. In certain aspects of the disclosure, in order to deposit in the alveolar airways, droplets in this size range must have momentum that is sufficiently high to permit ejection out of the droplet delivery device, but sufficiently low to overcome deposition onto the tongue (soft palate) or pharynx.

In certain embodiments, methods for generating an ejected stream of droplets from a fluid composition comprising nicotine for delivery to the respiratory system of user are provided. In certain embodiments, the ejected stream of droplets is generated in a controllable and defined droplet size range. By way of example, the droplet size range includes at least about 50%, at least about 60%, at least about 70%, at least about 85%, at least about 90%, between about 50% and about 90%, between about 60% and about 90%, between about 70% and about 90%, etc., of the ejected droplets are in the respirable range of below about 5 μm, below about 4 μm, below about 3.7 μm, below about 3.5 μm, below about 3.2 μm, below about 3.0 μm, below about 2 μm, between about 0.7 μm and about 4 μm, between about 0.7 μm and about 3.2 μm, between about 0.7 μm and about 3 μm, between about 0.7 μm and about 2.5 μm, between about 0.7 μm and about 2.0 μm, between about 0.7 μm and about 1.5 μm, between about 0.7 μm and about 1.0 μm, etc.

In other embodiments, the ejected stream of droplets may have one or more diameters, such that droplets having multiple diameters are generated so as to target multiple regions in the airways (mouth, tongue, throat, upper airways, lower airways, deep lung, etc.) By way of example, droplet diameters may range from about 0.7 μm to about 200 μm, about 0.7 μm to about 100 μm, about 0.7 μm to about 60 μm, about 0.7 μm to about 40 μm, about 0.7 μm to about 20 μm, about 0.7 μm to about 5μm, about 0.7 μm to about 4.7 μm, about 0.7 μm to about 4 μm, about 0.7 μm to about 3.0 μm, about 0.7 μm to about 2.5 μm, about 0.7 μm and about 2.0 μm, about 0.7 μm and about 1.5 μm, about 0.7 μm and about 1.0 μm, about 5 μm to about 20 μm, about 5 μm to about 10 μm, and combinations thereof. In particular embodiments, at least a fraction of the droplets has diameters in the respirable range, while other droplets may have diameters in other sizes so as to target non-respirable locations (e.g., larger than about 5 μm). Illustrative ejected droplet streams in this regard might have 50%-70% of droplets in the respirable range (less than about 5 μm), and 30%- 50% outside of the respirable range (about 5 μm-about 10 μm, about 5 μm-about 20 μm, etc.)

In some embodiments embodiment, methods for delivering safe, suitable, and repeatable dosages of a fluid composition comprising nicotine to the respiratory system of a user are provided herein. The methods of such embodiments may deliver an ejected stream of droplets to the desired location within the respiratory system of the user. In certain embodiments, the methods may be capable of delivering a defined volume of fluid in the form of an ejected stream of droplets such that an adequate and repeatable high percentage of the droplets are delivered into the desired location within the airways, e.g., the alveolar airways of the user during use.

In some embodiments, methods herein may include delivering a fluid composition comprising nicotine as an ejected stream of droplets in a respirable range to the respiratory system of a user. In certain embodiments, the method comprises (a) generating an ejected stream of droplets from the fluid composition via a droplet delivery device, wherein at least about 50% of the ejected stream of droplets have an average ejected droplet diameter of less than about 6 μm; and (b) delivering the ejected stream of droplets to the respiratory system of the user such that at least about 50% of the mass of the ejected stream of droplets is delivered in a respirable range to the respiratory system of a user during use.

In certain embodiments, the methods of the disclosure may be used to treat various diseases, disorders and conditions, promote or regulate various physiological activities, and combinations thereof, by delivering a fluid composition comprising nicotine to the respiratory system of a user. In this regard, the methods of the disclosure may be used to deliver nicotine locally to the respiratory system, and/or systemically to the body.

In accordance with the disclosure, any suitable droplet delivery device may be used in connection with the the disclosure. By way of example, droplet delivery devices that may be used with the methods described herein include, but are not limited to, those described in PCT/US2017/030913 (WO2017/192767), PCT/2017/030917 (WO2017/192771), PCT/2017/030919 (WO2017/192773), PCT/US2017/030921 (WO2017/192774), PCT/US2017/030929 (WO2017/192782), PCT/2017/030925 (WO2017/192778), PCT/US2018/054417 (WO 2019/071008), PCT/US2018/056300 (WO 2019/079461, PCT/2018/059874 (WO2019/094628), PCT/2019/012691 (WO2019/136437), PCT/US2019/25321 (WO2019/195239), PCT/2019/054042 (WO2020/072478), PCT/US2020/014785 (WO2020/154497), PCT/US2020/032383 (WO2020/227717), PCT/US2020/040132 (WO2020/264501), PCT/US2022/035492, PCT/US2022/026176, PCT/US2022/034552, U.S. patent application Ser. No. 17/846,902, U.S. Provisional Patent Application No. 63/256,245, U.S. Provisional Patent Application No. 63/318,202, U.S. Provisional Patent Application No. 63/323,770, U.S. Provisional Patent Application No. 63/346,794, U.S. Provisional Patent Application No. 63/337,885, U.S. Provisional Patent Application No. 63/390,170, U.S. Provisional Patent Application No. 63/390,209, and U.S. Provisional Patent Application No. 63/390,228, the disclosures of which are each incorporated herein by reference in their entirety.

In certain embodiments, compositions disclosed herein may be used with a “push mode” droplet delivery device that preferably does not include a heating requirement that could result in undesirable byproducts and comprises: a container assembly with an mouthpiece port; a reservoir disposed within or in fluid communication with the container assembly to supply a volume of fluid of the composition, an ejector bracket in fluid communication with the reservoir, the ejector bracket including a mesh with a membrane operably coupled to an electronic transducer (such as am ultrasonic transducer preferably including piezoelectric material) with the membrane between the transducer and the mesh, wherein the mesh includes a plurality of openings formed through the mesh's thickness, and wherein the transducer is coupled to a power source and is operable to oscillate the membrane and generate an ejected stream of droplets of composition through the mesh, and an ejection channel within the container assembly configured to direct the ejected stream of droplets from the mesh to the outlet. The vibrating membrane “pushing” liquid composition through the mesh is referred to as “push mode” ejection and devices in embodiments of the push mode invention may be referred to as push mode devices. A non-limiting example of such a device is described in U.S. patent application Ser. No. 17/846,902 and PCT/US2022/034552, the disclosures of which are each incorporated herein by reference in their entirety.

In one embodiment, the droplet delivery device may be configured to provide for ejection of droplets after a breath initiation period, e.g., 0.1-0.5 seconds. The device may be configured to sense the initiation of the inspiration cycles, allowing a short period of time, e.g., 0.1-0.5 seconds as to form a steady inspiration flow. Once the device senses a steady inspiration flow, the device may activate an ejector mechanism to initiate ejection of the small droplets for inhalation into the target site of the respiratory system. Optionally, the device may control the ejector mechanism to discontinue generation of droplets at a specified end portion of the inspiration cycle, so as to allow for complete inhalation of the droplets to the target site of the respiratory system. Such a device provides for an improved method of delivering droplets to the respiratory system of a user with minimal or no mouth or throat irritation.

In certain embodiments, methods herein including generating an ejected stream of droplets from a fluid composition comprising nicotine via an droplet delivery device comprising an ejector mechanism having an aperture plate (or mesh), the aperture plate (or mesh) having a plurality of openings formed through its thickness wherein the droplet delivery device is configured to directly or indirectly oscillate the aperture plate (or mesh) at a frequency to thereby generate the ejected stream of droplets, wherein at least about 50% of the ejected stream of droplets have an average ejected droplet diameter of less than about 6 μm; and delivering the ejected stream of droplets to the respiratory system of the user such that at least about 50% of the mass of the ejected stream of droplets is delivered in a respirable range to the respiratory system of a user during use.

In certain embodiments, the ejected stream of droplets of a fluid composition comprising nicotine herein may be generated via an ejector mechanism configured to provide coordinated and precise control of droplet size. In certain embodiments, the ejector mechanism of the droplet delivery device may comprise at least one aperture plate (or mesh) with a plurality of openings formed through its thickness for ejecting droplets, wherein at least one surface of the aperture plate (or mesh) is configured to provide a desired surface contact angle. In certain embodiments, the aperture plate (or mesh) may be configured such that at least one surface is configured with a desired surface contact angle to facilitate generation of droplets with the desired droplet size distribution, e.g., less than 4 μm, less than about 3.2 microns, less than about 3 microns, less than about 2 microns, less than about 1.5 microns, less than about 1 micron, etc.

In certain embodiments, the aperture plate (or mesh) has a plurality of openings formed through its thickness and at least the fluid entrance side of one or more of said plurality of openings configured so as to provide a surface contact angle of less than 90 degrees. In certain embodiments, at least about 50% of the droplets have an average ejected droplet diameter of less than about 6 microns during use. In some embodiments, at least a portion of the interior of at least one of the openings near the fluid entrance side is configured so as to provide a surface contact angle of less than 90 degree.

In other embodiments, the aperture plate (or mesh) is configured such that at least the fluid exit side of one or more of said plurality of openings is configured to provide a surface contact angle of greater than 90 degrees. In some embodiments, at least a portion of the interior of at least one of the openings near the fluid exit side is configured so as to provide a surface contact angle of greater than 90 degrees.

In certain embodiments, at least the fluid entrance surface of one or more openings of the aperture plate (or mesh) and the fluid exit surface of one or more openings of the aperture plate (or mesh) are configured (e.g., treated, coated, surface modified, or a combination thereof) to provide a desired surface contact angle. In some embodiments, at least a portion of the interior of at least one of the openings near the fluid entrance side is configured so as to provide a desired surface contact angle. By way of example, the fluid entrance surface and/or interior surface of one or more openings of the aperture plate (or mesh) may be configured to have a surface contact angle of less than about 80 degrees, less than about 70 degrees, less than about 50 degrees, less than about 55 degrees, less than about 50 degrees, less than about 40 degrees, less than about 35 degrees, less than about 30 degrees, less than about 20 degrees, less than about 10 degrees, between about 10 degrees and about 80 degrees, between about 10 degrees and about 60 degrees, between about 20 degrees and about 55 degrees, between about 10 and about 35 degrees, between about 15 and about 35 degrees, etc. By way of a further example, the fluid exit surface and/or interior surface of one or more openings of the aperture plate (or mesh) may be configured to have a surface contact angle of greater than greater than 90 degrees, between 90 degrees and 140 degrees, between 90 degrees and 135 degrees, between 100 degrees and 140 degrees, between 100 degrees and 135 degrees, between 90 degrees and 110 degrees, etc.

In certain aspects, the droplet delivery device is capable of delivering a defined volume of fluid (fixed dose) in the form of an ejected stream of droplets having a small average ejected diameter such that an adequate and repeatable high percentage of the droplets are delivered into the desired location within the airways, e.g., the alveolar airways of the user during use. In certain embodiments, the average droplet diameters may range from about 0.7 μm to about 5 μm, about 0.7 μm to about 4.7 μm, about 0.7 μm to about 4 μm, about 0.7 μm to about 3.2 μm, about 0.7 μm to about 2.5 μm, about 0.7 μm to about 1.3 μm, etc. In certain embodiments, the average droplet diameters may be less than about 4 microns, less than about 3.2 microns, less than about 3 microns, less than about 2 microns, less than about 1.5 microns, less than about 1 micron, etc. In certain embodiments, the average droplet diameters may range from about 1 μm to about 2 μm (e.g., about 1.0, 1.1, 1.2, 1.3, 1.4, 1.5, 1.6, 1.7, 1.8, 1.9, 2.0 μm). In certain embodiments, the average droplet diameters may range from about 3 μm to about 4 μm (e.g., about 3.0, 3.1, 3.2, 3.3, 3.4, 3.5, 3.6, 3.7, 3.8, 3.9, 4.0 μm). One of skill in the art can appreciate that the average droplet diameters may be optimized to meet clinical need while staying within an acceptable respirable range.

In certain embodiments, one or more surfaces of the aperture plate (or mesh) may be modified, treated, coated, or a combination thereof to achieve the desired surface contact angle. In certain aspects, the one or more surfaces of the aperture plate (or mesh) may be modified, treated, coated, or a combination thereof so as to affect surface hydrophobicity. By way of examples, one or more surfaces of the aperture plate (or mesh) may be modified, treated, coated, or a combination thereof so as to result in at least one more hydrophilic surface on the aperture plate (or mesh), optionally at least one more hydrophobic surface on the aperture plate (or mesh), or a combination thereof. In certain embodiments, at least the fluid entrance side, and optionally the fluid exit surface side are configured so as to have a desired surface contact angle. In certain embodiments, at least a portion of the interior surface of one or more openings may be configured so as to have a desired surface contact angle.

In addition to aperture plate (or mesh) surface contact angle, several features of the ejector mechanism allow for precise dosing of specific droplet sizes. For instance, droplet size is set, in part, by the diameter of the openings in the aperture plate (or mesh), which are formed with high accuracy. By way of example, the openings in the aperture plate (or mesh) at the fluid exit side of the aperture plate (or mesh) may range in size from 1 μm to 6 μm, from 2 μm to 5 μm, from 3 μm to 5 μm, from 3 μm to 4 μm, about 1.7 μm, about 2.0 μm, about 3.5 μm, about 3.9 μm, etc. In certain embodiments, the aperture plate (or mesh) may include openings having different cross-sectional shapes or diameters to thereby provide ejected droplets having different average ejected droplet diameters. Ejector rate also influences droplet size. Ejection rate, in droplets per second, is fixed by the frequency of the aperture plate (or mesh) vibration, e.g., 108-kHz, etc.

In certain aspects of the disclosure, desired surface contact angles may be formed by creating hydrophilic surfaces, e.g., through treating, coating, surface modifying, or a combination thereof. A surface is considered to be hydrophilic when that angle is less than about 80 degrees, about 70 degrees, about 60 degrees, about 55 degrees, about 50 degrees, etc., and may be considered to be super hydrophilic when that angle is less than about 10 to 20 degrees (droplet tends to spread out across the surface). The strength of the hydrophilic effect may be measured by the angle between the edge of a droplet of water and the surface of the aperture plate (or mesh).

By way of example, the aperture plate (or mesh) can be formed of a metal, e.g., stainless steel, nickel, cobalt, titanium, iridium, platinum, or palladium or alloys thereof, and configured to achieve the desired contact angles as described herein. Alternatively, the aperture plate (or mesh) can be formed of suitable polymeric material and be configured to achieve the desired surface contact angles, as described herein. By way of example, the aperture plate (or mesh) may be composed of a material selected from the group consisting of polyethylene naphthalate (PEN), polyetheretherketone (PEEK), polyimide, polyetherimide, polyvinylidine fluoride (PVDF), ultra-high molecular weight polyethylene (UHMWPE), polysulfone, nickel, nickel-cobalt, nickel-palladium, palladium, platinum, metal alloys thereof, and combinations thereof. Further, in certain aspects, the aperture plate (or mesh) may comprise a domed shape.

By way of example, the desired surface contact angle may be created on a surface of an aperture plate (or mesh) by increasing the surface energy through creation of a polar surface. Exemplary methods to increase surface energy comprise forming an oxide surface on a metallic ejector aperture plate (or mesh) which is polar. In accordance with aspects of the disclosure, exemplary methods for creating a hydrophilic surface contact angle on an aperture plate (or mesh) including dip coating methods, etching methods, and chemical deposition methods. Dip coating methods comprise dipping the aperture plate (or mesh) into a solution comprising a desired coating and a solvent, which solution will form a hydrophilic coating on the surface when the solvent evaporates. Chemical deposition methods include known deposition methods, e.g., plasma etch, plasma coating, plasma deposition, CVD, electroless plating, electroplating, etc., wherein the chemical deposition uses a plasma or vapor to open the bonds on the surface of the aperture plate (or mesh) so that oxygen or hydroxyl molecules attach to the surface rendering it polar. Etching methods include non-chemical etching methods using surface roughening.

In certain embodiments, any deposited hydrophilic layer is significantly thinner than the opening size such that it does not impact the size of the generated droplets. In certain embodiments, the surface treatment may extend into at least a portion of one or more openings of the aperture plate (or mesh) so as to form a hydrophilic surface within at least a portion of one or more openings.

In certain embodiments, the desired surface contact angle may be obtained through surface roughening achieved, e.g., via non-chemical etching. Without intending to be limited by theory, as an approximation, the Wenzel Contact Angle equation, “Apparent Contact Angles on Rough Surfaces: the Wenzel Contact Angle Revisited”, Wolansky and Marmur, Colloids and Surfaces A, 156 (1999) pp. 381-388, may be used to estimate surface contact angle. The Wenzel equation yields contact angles for liquid drops on rough surfaces. It assumes no hysteresis in the contact angle, and this is an approximation.

In certain embodiments, the aperture plate (or mesh) may optionally be surface sputtered with a thin layer (e.g., about 30 to about 150 nm, about 60 nm to about 100 nm, about 30 nm, about 60 nm, about 80 nm, about 100 nm, etc. thick sputtering) of a precious metal, such as gold (Au), palladium (Pd), platinum (Pt), silver (Ag) and precious metal alloys. In certain embodiments, the surface may be sputtered with a thin layer of palladium. The precious metal layer may then be etched at varying etch powers, e.g., low, medium or high etching power to provide a desired surface contact angle. To provide the desired contact angle, the etch may be performed once, twice, three times, four times, etc.

In other embodiments, the aperture plate (or mesh) may be coated on at least the fluid entrance side of the aperture plate (or mesh) with a hydrophilic polymer to achieve the desired surface contact angle. In yet other embodiments, the aperture plate (or mesh) may be coated on at least a portion of the interior surface of one or more openings, within the entire interior surface of one or more openings, on both the fluid entrance side and the fluid ejection surface of the aperture plate (or mesh), and combinations thereof. Any known hydrophilic polymer suitable for use in medical applications may be used.

Any suitable hydrophilic coating to achieve the desired surface contact angle on the fluid entrance side of the ejector aperture plate (or mesh) may be used. Exemplary hydrophilic coating materials include, but are not limited to siloxane based coatings, isocyante based coatings, ethylene oxide based coatings, polyisocyanate based coatings, hydrocyclosiloxane based coatings, hydroxyalkylmethacrylate based coatings, hydroxyalkylacrylate based coatings, glycidylmethacrylate based coatings, propylene oxide based coatings, N-vinyl-2-pyrrolidone based coatings, latex based coatings, polyvinylchloride based coatings, polyurethane based coatings, etc.

By way of non-limiting example, a suitable hydrophilic coating may comprise a single layer hydrophilic surface formed by a process of cleaning the intended surface with a low pressure plasma and then dipping the surface into a solution of organophosphorous acids which self-assemble into a polar monolayer (e.g., see Aculon U.S. Pat. No. 8,658,258A, which is incorporated herein by reference). These layers are typically less than 10 nm thick, which is significantly less than a micron-sized hole. Contact angles as low as 10 degrees can be achieved using such coatings.

In other embodiments, the aperture plate (or mesh) may optionally be coated on the fluid exit side with a hydrophobic coating. Any known hydrophobic polymer suitable for use in medical applications may be used, e.g., polytetrafluoroethylene (Teflon), siloxane based coatings, paraffin, polyisobutylene, polysulfone, etc. The surface of the hydrophobic coating may be chemically or structurally modified or treated to further enhance or control the surface contact angle, as desired.

In certain embodiments, the aperture plate (or mesh) may be coated with a siloxane based coating to provide an initial hydrophobic coating, which siloxane based coating is thereafter masked or shielded in a suitable manner on the fluid exit side. Following masking, the masked aperture plate (or mesh) may thereafter be exposed to an oxidizing treatment to render the siloxane coating hydrophilic on the exposed (unmasked) portions thereof, i.e., the fluid entrance sides. In this manner, in certain embodiments of the disclosure, the same siloxane based coating may provide both hydrophilic and hydrophobic coatings to surfaces of the aperture plate (or mesh). By way of example, such siloxane coatings may be selected from siloxanes known for use in medical applications, such as 2,4,6,8-Tetramethylcyclotetrasiloxane, or 1,1,3,3-Tetramethyldisiloxane.

The aperture plate (or mesh) may be metallic or polymer with openings about the diameter of the desired droplets (as discussed further herein). By way of non-limiting example, the aperture plate (or mesh) may be formed from silicon, silicon carbide, nickel palladium, or a high stiffness polymer such as polyetheretherketone (PEEK), poly-amide, Kapton or Ultra High Molecular Weight Polyethylene (UHMWPE). When using a polymer aperture plate (or mesh), the openings may be produced by rolling, stamping, laser ablation, bulk etching or other known micro-machining processes. When using silicon and SiC for the aperture plate (or mesh), the openings may be formed using typical semiconductor processes. Without being limited, these silicon materials can be formed by bulk micro-machining processes, such as wet etching. In addition, the aperture plate (or mesh) opening area may be formed to have a dome-like shape to increase the stiffness of the aperture plate (or mesh) and to creating uniform ejection accelerations.

The aperture plate (or mesh) may have an array of opening ranging from, e.g., 100 to 10,000 openings, 500 to 10,000 openings, etc. The openings may generally have a fluid exit side diameter similar to that of the desired droplets, e.g., of 0.5 μm to 100 μm diameter, 1 μm to 20 μm, 1 μm to 10 μm, 1 μm to 5 μm, 1 μm to 4 μm, etc., as described further herein. The fluid entrance side diameter may range from between about 30 μm to 300 μm, about 75 μm to about 200 μm, about 100 μm to about 200 μm, etc. Aperture plate (or mesh) s may be formed to have a thickness of between about 100 μm to about 925 μm, between about 100 μm and about 300 μm.

As described above, the aperture plate (or mesh) may include various treatments, coatings surface modifications, or combinations thereof, on one or more surfaces thereof. For example, in certain embodiments, the aperture plate (or mesh) may include various combinations of a hydrophilic coating on one or more surfaces, an optional hydrophobic coating on one or more surfaces, native surfaces, surface etchings, etc. In one embodiment, the aperture plate (or mesh) may be non-chemically etched on the fluid entrance side of the aperture plate (or mesh) (fluid reservoir facing side), with etching, a hydrophobic coating, or no treatment on the fluid exit side. In another embodiment, the aperture plate (or mesh) may include a hydrophilic coating on at least the fluid entrance side of the aperture plate (or mesh) (fluid reservoir facing side), a hydrophilic coating within at least a portion of the interior of one or more openings, or combinations thereof. In other embodiments, the aperture plate (or mesh) may include a hydrophobic coating on the droplet exit side of the aperture plate (or mesh)-alone or in combination with one or more hydrophilic coatings. A gas or liquid process may be used to form the hydrophobic and hydrophilic surfaces. For example, hydrophilic and hydrophobic surfaces can be formed using liquid coating, sputtering, CVD, plasma deposition, ion implantation, etc.

The aperture plate (or mesh) s may be produced, e.g., by semiconductor techniques, stamping, rolling or laser ablation. Rolling may be preferred because more precise forming pressures are possible and continuous production for material from rolls allows lower-cost manufacturing. Because the material stiffness of polymers (especially the UHMWPE) is lower than metals such as stainless steel or palladium-nickel, ribs on the fluid or air side of the aperture plate (or mesh) may also be formed at the time of rolling or prior to laser ablation. Similarly, a metallic annulus may be used to stiffen the edge of the aperture plate (or mesh) against flexure. In addition, the aperture plate (or mesh) area can be formed to have a dome-like shape to increase the stiffness of the aperture plate (or mesh) and creating uniform ejection accelerations.

All publications and patent applications cited in this specification are herein incorporated by reference as if each individual publication or patent application were specifically, and individually, indicated to be incorporated by reference.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of embodiments of the inventions.

### Example 1

In one exemplary method, a formulation containing nicotine was prepared. The formula contained 2% (w/w) Nicotine, with 1.75%±0.5 (w/w) L-(+)-lactic acid (Cas. #79-33-4) in Type I water. The formula had a pH of about 5.2.

### Example 2

In one exemplary method, a formulation containing nicotine was prepared. The formula contained 2% (w/w) Nicotine, with 1.75%±0.5 (w/w) L-(+)-lactic acid (Cas. #79-33-4), 10% (w/w) ethanol, 0.088% (w/w) L-Menthol (Cas #2216-51-5) in type I water. The formula had a pH of about 5.2.

Various embodiments of the invention have been described. It will, however, be evident that various modifications and changes may be made thereto, and additional embodiments may be implemented, without departing from the broader scope of the invention as set forth in this disclosure. This specification is to be regarded in an illustrative rather than a restrictive sense.

What is claimed is:

1. A composition comprising:
nicotine in a concentration of about 0.1% (w/w) to about 5% (w/w);
L-lactic acid in a concentration of about 0.1% (w/w) to about 5% (w/w); and
water.

2. The composition of claim 1, wherein the nicotine is in a concentration of about 1.6% to about 2% (w/w).

3. The composition of claim 2, wherein the L-lactic acid is in a concentration of about 1.2% to about 1.8% (w/w).

4. The composition of claim 1, wherein the L-lactic acid is in a concentration of about 1.2% to about 1.8% (w/w).

5. The composition of claim 4, wherein the composition has a pH of about 5.2+/−0.5.

6. The composition of claim 3, wherein the composition has a pH of about 5.2+/−0.5.

7. The composition of claim 2, wherein the composition has a pH of about 5.2+/−0.5.

8. The composition of claim 1, wherein the composition has a pH of about 5.2+/−0.5.

9. The composition of claim 8, wherein the composition is substantially free of D-lactic acid.

10. The composition of claim 7, wherein the composition is substantially free of D-lactic acid.

11. The composition of claim 6, wherein the composition is substantially free of D-lactic acid.

12. The composition of claim 5, wherein the composition is substantially free of D-lactic acid.

13. The composition of claim 4, wherein the composition is substantially free of D-lactic acid.

14. The composition of claim 3, wherein the composition is substantially free of D-lactic acid.

15. The composition of claim 2, wherein the composition is substantially free of D-lactic acid.

16. The composition of claim 1, wherein the composition is substantially free of D-lactic acid.

17. The composition of any of claims 1-16, wherein the composition further comprises ethanol.

18. The composition of claim 17, wherein the ethanol is at a concentration of less than or equal to about 10% (w/w).

19. The composition of any of claims 1-16, wherein the composition further comprises menthol.

20. The composition of claim 19, wherein the menthol is at a concentration of less than or equal to about 0.09% (w/w).

21. The composition of claim 20, wherein the menthol is L-menthol.

22. The composition of claim 19, wherein the menthol is L-menthol.

23. The composition of any of claims 1-16, wherein the composition further comprises propylene glycol.

24. The composition of claim 23, wherein the propylene glycol is at a concentration of less than or equal to about 15% (w/w).

25. The composition of any of claims 1-16, wherein the composition further comprises cyclodextrin.

26. The composition of claim 25, wherein the cyclodextrin is at a concentration of less than or equal to about 5% (w/w).

27. The composition of any of claims 1-16, wherein the composition further comprises WS-23 (2-isopropyl-N,2,3-trimethylbutyramide).

28. The composition of claim 27, wherein the cyclodextrin is at a concentration of less than or equal to about 0.5% (w/w).

29. The composition of any of claims 1-16, wherein the composition further comprises one or more concentrated fruit juices.

30. The composition of claim 29, wherein the one or more concentrated fruit juices are at a concentration of less than or equal to about 5% (w/w).

31. The composition of any of claims 1-16, wherein the composition is in the form of droplets and suitable for delivery to the respiratory system of a subject.

32. A droplet delivery device container including the composition of any claims 1-16 configured to dispense the composition to a droplet delivery device that produces a stream of droplets suitable for delivery to the respiratory system of a subject.

33. The droplet delivery device of claim 32, further comprising a power source, an electronic actuator, and a membrane between the electronic actuator and an aperture plate with a plurality of openings that ejects the stream of droplets.

34. The droplet delivery device of claim 33, wherein the device is free of a heating element.

35. The droplet delivery device of claim 32, wherein the device is free of a heating element.

36. The droplet delivery device of claim 35, wherein the stream of droplets produced by the droplet has a mass median aerodynamic diameter of less than or equal to about 3 μm.

37. The droplet delivery device of claim 34, wherein the stream of droplets produced by the droplet has a mass median aerodynamic diameter of less than or equal to about 3 μm.

38. The droplet delivery device of claim 33, wherein the stream of droplets produced by the droplet has a mass median aerodynamic diameter of less than or equal to about 3 μm.

39. The droplet delivery device of claim 32, wherein the stream of droplets produced by the droplet has a mass median aerodynamic diameter of less than or equal to about 3 μm.

40. The droplet delivery device of claim 39, wherein the device is configured to deliver the stream of droplets produced by the composition during peak flow of a user's inhalation cycle to minimize mouth or throat irritation.

41. The droplet delivery device of claim 39, wherein the device is configured to deliver the stream of droplets produced by the composition during peak flow of a user's inhalation cycle to minimize mouth or throat irritation.

42. The droplet delivery device of claim 38, wherein the device is configured to deliver the stream of droplets produced by the composition during peak flow of a user's inhalation cycle to minimize mouth or throat irritation.

43. The droplet delivery device of claim 37, wherein the device is configured to deliver the stream of droplets produced by the composition during peak flow of a user's inhalation cycle to minimize mouth or throat irritation.

44. The droplet delivery device of claim 36, wherein the device is configured to deliver the stream of droplets produced by the composition during peak flow of a user's inhalation cycle to minimize mouth or throat irritation.

45. The droplet delivery device of claim 35, wherein the device is configured to deliver the stream of droplets produced by the composition during peak flow of a user's inhalation cycle to minimize mouth or throat irritation.

46. The droplet delivery device of claim 34, wherein the device is configured to deliver the stream of droplets produced by the composition during peak flow of a user's inhalation cycle to minimize mouth or throat irritation.

47. The droplet delivery device of claim 33, wherein the device is configured to deliver the stream of droplets produced by the composition during peak flow of a user's inhalation cycle to minimize mouth or throat irritation.

48. The droplet delivery device of claim 32, wherein the device is configured to deliver the stream of droplets produced by the composition during peak flow of a user's inhalation cycle to minimize mouth or throat irritation.

49. The droplet delivery device of claim 48, wherein the device is configured to discontinue generation of droplets at a specified end portion of the inspiration cycle.

50. The droplet delivery device of claim 32, wherein the device is configured to discontinue generation of droplets at a specified end portion of the inspiration cycle.

51. The droplet delivery device of claim 33, wherein the device is configured to discontinue generation of droplets at a specified end portion of the inspiration cycle.

52. The droplet delivery device of claim 39, wherein the device is free of a heating element.

53. A method of providing smoking cessation therapy comprising administering a stream of droplets produced by the composition of any of claims 1-16.

54. A method of providing smoking cessation therapy comprising administering a stream of droplets produced by the composition of claim 17.

55. A method of providing smoking cessation therapy comprising administering a stream of droplets produced by the composition of claim 19.

56. A method of providing smoking cessation therapy comprising administering a stream of droplets produced by the composition of claim 23.

57. A method of providing smoking cessation therapy comprising administering a stream of droplets produced by the composition of claim 25.

58. A method of providing smoking cessation therapy comprising administering a stream of droplets produced by the composition of claim 27.

59. A method of providing smoking cessation therapy comprising administering a stream of droplets produced from the composition by the droplet delivery device of claim 32.

60. A droplet delivery device free of a heating element and comprising a reservoir that supplies a composition including nicotine in a concentration of about 0.1% (w/w) to about 5% (w/w), L-lactic acid in a concentration of about 0.1% (w/w) to about 5% (w/w), and water.

61. A droplet delivery device comprising a reservoir that supplies a composition including nicotine in a concentration of about 0.1% (w/w) to about 5% (w/w), L-lactic acid in a concentration of about 0.1% (w/w) to about 5% (w/w), and water, wherein the device produces droplets from the composition with a mass median aerodynamic diameter in the respirable range of less than 6 μm.

62. The droplet delivery device of claim 61, wherein the device is free of a heating element.
